(19) United States
(12) Patent Application Publication
(10) Pub. No.: US 2025/0067697 A1
(43) Pub. Date: Feb. 27, 2025

(54) SENSOR

(71) Applicant: KABUSHIKI KAISHA TOSHIBA, Tokyo (JP)

(72) Inventors: Yoshihiko KURUI, Chigasaki Kanagawa (JP); Hiroaki YAMAZAKI, Yokohama Kanagawa (JP)

(73) Assignee: KABUSHIKI KAISHA TOSHIBA, Tokyo (JP)

(21) Appl. No.: 18/760,501

(22) Filed: Jul. 1, 2024

(30) Foreign Application Priority Data
Aug. 24, 2023 (JP) 2023-136487

(51) Int. Cl. G01N 27/18; G01N 33/00

(52) U.S. Cl. CPC G01N 27/18; G01N 33/0027

(57) ABSTRACT

According to one embodiment, a sensor includes a sensor section and a circuit section. The sensor section includes an element portion including a sensor element and a conductive member. The circuit section includes a first differential circuit, a voltage holding circuit, a first switch, a second switch, a third switch, and a controller. The controller is configured to perform a first operation and a second operation. In the first operation, the controller is configured to set the third switch to a third connected state, to set the first switch to a first connected state, and to set the second switch to a second disconnected state. In the second operation, the controller is configured to set the third switch to a third disconnected state, to set the first switch to a first disconnected state, and to set the second switch to a second connected state.

### CROSS-REFERENCE TO RELATED APPLICATIONS

This application is based upon and claims the benefit of priority from Japanese Patent Application No. 2023-136487, filed on Aug. 24, 2023; the entire contents of which are incorporated herein by reference.

### FIELD

Embodiments described herein generally relate to a sensor.

### BACKGROUND

For example, there are sensors using MEMS (Micro Electro Mechanical Systems) elements. It is desired to improve the characteristics of sensors.

### BRIEF DESCRIPTION OF THE DRAWINGS

FIG. 1 is a block diagram illustrating a sensor according to a first embodiment;

FIG. 2 is a schematic cross-sectional view illustrating the sensor according to the first embodiment;

FIG. 3 is a block diagram illustrating the operation of the sensor according to the first embodiment;

FIG. 4 is a block diagram illustrating the operation of the sensor according to the first embodiment;

FIG. 5 is a block diagram illustrating the operation of the sensor according to the first embodiment;

FIG. 6 is a time chart illustrating the operation of the sensor according to the first embodiment;

FIG. 7 is a block diagram illustrating a sensor according to a second embodiment;

FIG. 8 is a block diagram illustrating the operation of the sensor according to the second embodiment;

FIG. 9 is a block diagram illustrating the operation of the sensor according to the second embodiment;

FIG. 10 is a block diagram illustrating the operation of the sensor according to the second embodiment;

FIG. 11 is a schematic cross-sectional view illustrating the sensor according to the embodiment;

FIG. 12 is a schematic plan view illustrating the sensor according to the embodiment; and

FIG. 13 is a schematic plan view illustrating the sensor according to the embodiment.

### DETAILED DESCRIPTION

According to one embodiment, a sensor includes a sensor section and a circuit section. The sensor section includes an element portion including a sensor element and a conductive member. The circuit section includes a first differential circuit, a voltage holding circuit, a first switch, a second switch, a third switch, and a controller. A sensor element end of the sensor element is electrically connected to a first current source. A sensor element other end of the sensor element is set to a first reference voltage. A first switch end of the first switch is electrically connected to a first connection point between the sensor element end and the first current source. A first switch other end of the first switch is electrically connected to an input end of the voltage holding circuit. An output end of the voltage holding circuit is electrically connected to a first input end of the first differential circuit. A second switch end of the second switch is electrically connected to the first connection point. A second switch other end of the second switch is electrically connected to a first other input end of the first differential circuit. A third switch end of the third switch is connected to a voltage source. A third switch other end of the third switch is electrically connected to a conductive member end of the conductive member. A conductive member other end of the conductive member is set to the first reference voltage. The controller is configured to perform a first operation and a second operation. In the first operation, the controller is configured to set the third switch to a third connected state, to set the first switch to a first connected state, and to set the second switch to a second disconnected state, and the voltage holding circuit is configured to hold a first connection point voltage of the first connection point in the first operation. In the second operation, the controller is configured to set the third switch to a third disconnected state, to set the first switch to a first disconnected state, and to set the second switch to a second connected state, and the first differential circuit is configured to output a first difference between the first connection point voltage in the second operation and a holding voltage held by the voltage holding circuit.

Various embodiments are described below with reference to the accompanying drawings.

The drawings are schematic and conceptual; and the relationships between the thickness and width of portions, the proportions of sizes among portions, etc., are not necessarily the same as the actual values. The dimensions and proportions may be illustrated differently among drawings, even for identical portions.

In the specification and drawings, components similar to those described previously in an antecedent drawing are marked with like reference numerals, and a detailed description is omitted as appropriate.

### First Embodiment

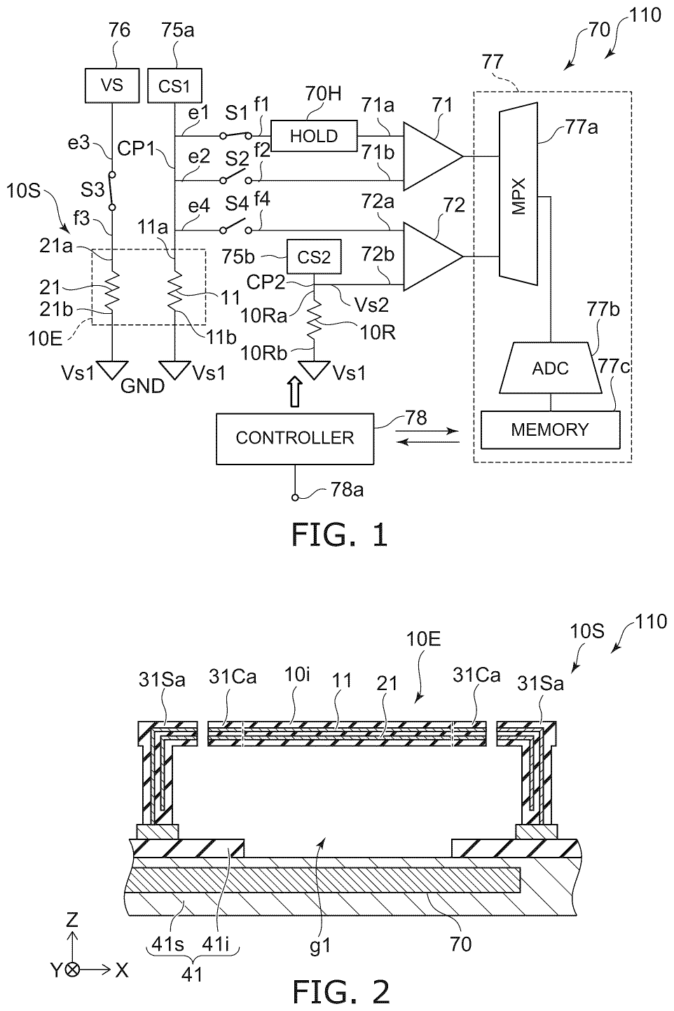

FIG. 1 is a block diagram illustrating a sensor according to a first embodiment.

FIG. 2 is a schematic cross-sectional view illustrating the sensor according to the first embodiment.

As shown in FIG. 1, a sensor 110 according to the embodiment includes a sensor section 10S and a circuit section 70. The sensor section 10S includes an element portion 10E. The element portion 10E includes a sensor element 11 and a conductive member 21.

FIG. 2 illustrates the sensor section 10S. For example, the sensor section 10S includes a base 41 and a first support portion 31Sa. The first support portion 31Sa is fixed to the base 41. The first support portion 31Sa support the element portion 10E. A first gap g1 is provided between the base 41 and the element portion 10E. In this example, the sensor section 10S includes a first opposing support portion 31Sb. The first opposing support portion 31Sb is fixed to the base 41. The first opposing support portion 31Sb supports the element portion 10E.

In this example, the sensor section 10S includes a first connect portion 31Ca and a first opposing connect portion 31Cb. The first connect portion 31Ca is supported by the first support portion 31Sa and supports the element portion 10E. The first opposing connect portion 31Cb is supported by the first opposing support portion 31Sb and supports the element portion 10E. As described below, these connect portions may have a meandering structure. Heat conduction through these connect portions is suppressed. At least a part of the sensor element 11 may overlap at least a part of the conductive member 21. An example of the configuration of the sensor section 10S will be described later.

In the embodiment, a voltage VS (see FIG. 1) is applied to the conductive member 21 in a pulsed manner. As a result, the temperature of the conductive member 21 increases. Along with this, the temperature of the sensor element 11 increases. At this time, the temperature of the sensor element 11 depends on the type and concentration of the detection target gas around the element portion 10E. This is due to the phenomenon that thermal conductivity (heat dissipation) changes depending on the type and concentration of the detection target gas. The temperature of the sensor element 11 when the heat dissipation is high is lower than the temperature of the sensor element 11 when the heat dissipation is low. A change in the temperature of the sensor element 11 can be detected as a change in the electrical resistance of the sensor element 11. For example, by detecting a change in the electrical resistance of the sensor element 11, the type and concentration of the detection target gas can be detected. The sensor 110 is, for example, a gas sensor. The sensor 110 is, for example, a thermally conductive gas sensor. The sensor element 11 is, for example, a resistance element.

There is a reference example in which a plurality of sensor sections 10S are used in order to obtain high accuracy in detecting the detection target gas. In the reference example, one sensor section 10S is used as a detection element, and another element is used as a reference element. In this case, the number of sensor sections 10S increases, making it difficult to downsize the sensor. Furthermore, it is difficult to obtain highly accurate detection results due to differences in characteristics that occur in the plurality of sensor sections 10S.

In the embodiment, one sensor section 10S is used. Using the sensor section 10S, a detection result is derived from the results obtained by a plurality of operations. This makes it easy to downsize the sensor. Furthermore, there is no deterioration in accuracy due to differences in characteristics that occur when the plurality of sensor sections 10S are used. Accurate detection results can be obtained. According to the embodiment, a sensor with improved characteristics can be provided.

An example of the configuration of the sensor section 10S and an example of the operation of the sensor 110 will be described below. As shown in FIG. 1, the circuit section 70 includes a first differential circuit 71, a voltage holding circuit 70H, a first switch S1, a second switch S2, a third switch S3, and a controller 78.

As shown in FIG. 1, the sensor element end 11a of the sensor element 11 is electrically connected to a first current source 75a (CS1). A sensor element other end 11b of the sensor element 11 is set to a first reference voltage Vs1. The first reference voltage Vs1 is, for example, a ground voltage (GND).

A first switch end e1 of the first switch S1 is electrically connected to a first connection point CP1 between the sensor element end 11a and the first current source 75a. A first switch other end f1 of the first switch S1 is electrically connected to an input end of the voltage holding circuit 70H. An output end of the voltage holding circuit 70H is electrically connected to a first input end 71a of the first differential circuit 71.

A second switch end e2 of the second switch S2 is electrically connected to the first connection point CP1. A second switch other end f2 of the second switch S2 is electrically connected to a first other input end 71b of the first differential circuit 71.

A third switch end e3 of the third switch S3 is connected to a voltage source 76 (voltage VS). A third switch other end f3 of the third switch S3 is electrically connected to a conductive member end 21a of the conductive member 21. A conductive member other end 21b of the conductive member 21 is set to the first reference voltage Vs1 (for example, GND).

As shown in FIG. 1, the circuit section 70 may include a processing circuit 77. For example, the processing circuit 77 may include a multiplexer 77a, an AD converter 77b, and a storage 77c. The multiplexer 77a is configured to, for example, multiplex the output of the first differential circuit 71 and the output of the second differential circuit 72. The AD converter 77b is configured to AD convert the output of the multiplexer 77a. The storage 77c is configured to store the output of the AD converter 77b.

The controller 78 may control the first differential circuit 71, the voltage holding circuit 70H, the first switch S1, the second switch S2, the third switch S3, and the processing circuit 77.

In the embodiment, the controller 78 is configured to perform a first operation and a second operation described below.

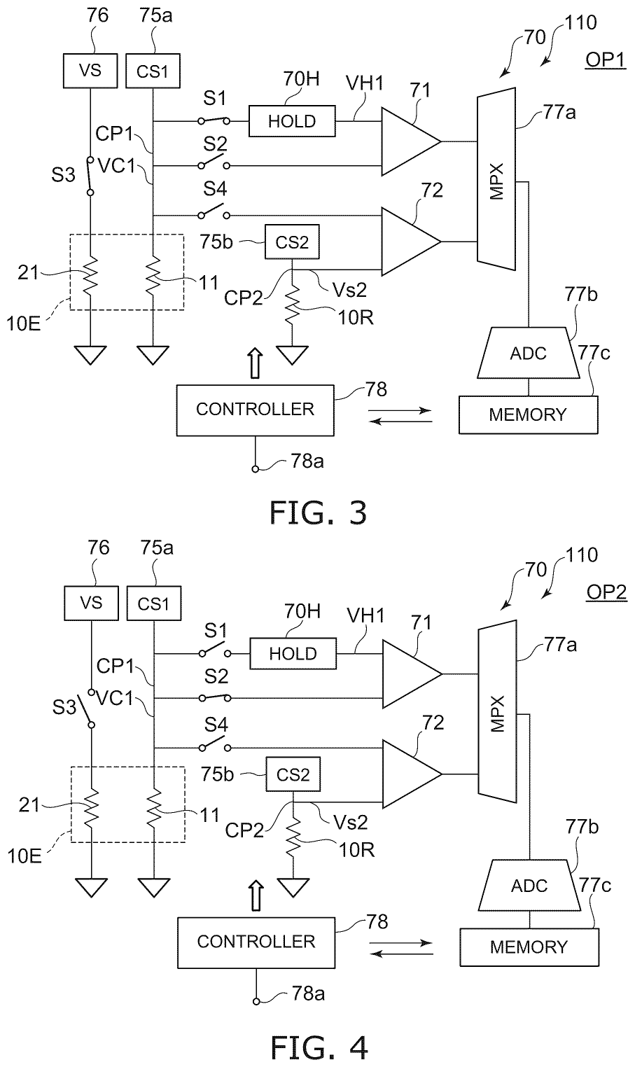

FIGS. 3 and 4 are block diagrams illustrating the operation of the sensor according to the first embodiment.

FIG. 3 illustrates the first operation. FIG. 4 illustrates the second operation.

As shown in FIG. 3, in the first operation OP1, the controller 78 sets the third switch S3 to a third connected state, sets the first switch S1 to a first connected state, and sets the second switch S2 to a second disconnected state. The voltage holding circuit 70H holds a first connection point voltage VC1 of the first connection point CP1 in the first operation OP1.

As shown in FIG. 4, in the second operation OP2, the controller 78 sets the third switch S3 to a third disconnected state, sets the first switch S1 to a first disconnected state, and sets the second switch S2 to a second connected state. The first differential circuit 71 outputs a first difference between the first connection point voltage VC1 in the second operation OP2 and the holding voltage VH1 held by the voltage holding circuit 70H.

The first operation OP1 corresponds to a detection operation, for example. The second operation OP2 corresponds to a reference value derivation operation, for example. A value corresponding to the electrical resistance of the sensor element 11 when a voltage is applied to the conductive member 21 and the temperature of the sensor element 11 is raised is derived by the first operation OP1. A value (reference value) corresponding to the electrical resistance of the sensor element 11 when no voltage is applied to the conductive member 21 is derived by the second operation OP2. The difference (first difference) between these values corresponds to the detection result corrected by the reference value. The first difference corresponds to, for example, the detection result of the concentration of the detection target gas. The first difference may correspond to, for example, the type of the detection target gas. According to the embodiment, a high accuracy detection result can be obtained by using one sensor section 10S.

In the embodiment, the value corresponding to the first difference may be stored in the storage 77c. In the embodiment, the value corresponding to the first connection point voltage VC1 may be stored in the storage 77c. In the embodiment, the value corresponding to the holding voltage VH1 may be stored in the storage 77c.

For example, the storage 77c included in the circuit section 70 is configured to store the first difference and the second difference. The circuit section 70 acquires the first difference and the second difference stored in the storage 77c, and can output a value obtained by correcting the first difference based on the second difference.

As shown in FIG. 1, the circuit section 70 may further include a second differential circuit 72 and a fourth switch S4. A fourth switch end e4 of the fourth switch S4 is electrically connected to the first connection point CP1. A fourth switch other end f4 of the fourth switch S4 is electrically connected to a second input end 72a of the second differential circuit 72. A second other input end 72b of the second differential circuit 72 is set to a second reference voltage Vs2.

For example, the second other input end 72b is electrically connected to a second connection point CP2. The second connection point CP2 is a connection point between a reference resistance element end 10Ra of the reference resistance element 10R and a second current source 75b (CS2). A reference resistance element other end 10Rb of the reference resistance element 10R is set to the first reference voltage Vs1 (e.g., GND). The second reference voltage Vs2 is generated by the second current source 75b and the reference resistance element 10R.

For example, the temperature dependence of the reference resistance element 10R is low. For example, the temperature dependence of the electrical resistance of the reference resistance element 10R is 10 ppm/° C. or less. By using the reference resistance element 10R, temperature compensation can be performed. For example, the controller 78 may further perform the following third operation.

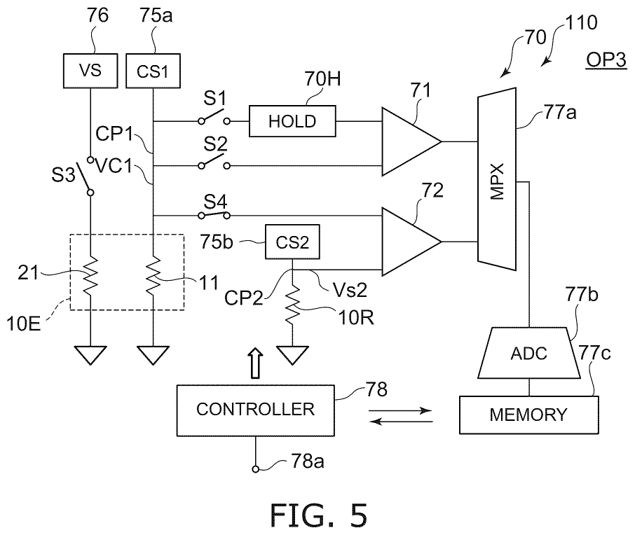

FIG. 5 is a block diagram illustrating the operation of the sensor according to the first embodiment.

FIG. 5 illustrates a third operation.

As shown in FIG. 5, in the third operation OP3, the controller 78 sets the third switch S3 to the third disconnected state, sets the first switch S1 to the first disconnected state, sets the second switch S2 to the second disconnected state, and sets the fourth switch S4 to a fourth connected state. The second differential circuit 72 outputs a second difference between the first connection point voltage VC1 and the second reference voltage Vs2 in the third operation OP3.

The second difference corresponds to a temperature measurement result using the reference resistance element 10R. The circuit section 70 may output a value obtained by correcting the first difference (detection result of the detection target gas) based on the second difference (measurement result of the temperature). For example, the concentration of the detection target gas is corrected by temperature. A detection result with higher accuracy is obtained.

The correction may be performed, for example, by correcting the value corresponding to the first difference stored in the storage 77c by the second difference. The temperature-corrected result may be stored in the storage 77c. The corrected result may be output from an output portion 78a of the controller 78, for example.

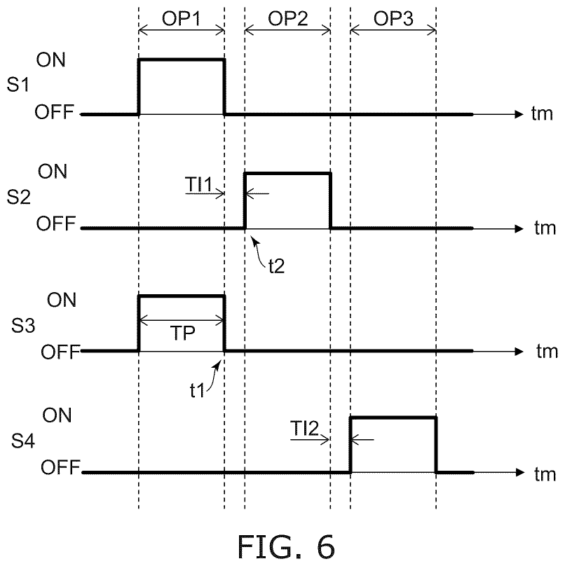

FIG. 6 is a time chart illustrating the operation of the sensor according to the first embodiment.

The horizontal axis of these figures is time tm.

As shown in FIG. 6, in the first operation OP1, the first switch S1 is ON (connected state), the second switch S2 is OFF (disconnected state), the third switch S3 is ON (connected state), and the fourth switch S4 is OFF (disconnected state). In the second operation OP2, the first switch S1 is OFF (disconnected state), the second switch S2 is ON (connected state), the third switch S3 is OFF (disconnected state), and the fourth switch S4 is OFF (disconnected state). In this example, the first operation OP1 is followed by the second operation OP2.

The ON time of the third switch S3 in the first operation OP1 corresponds to the pulse time TP of the voltage applied to the conductive member 21. The time between the first operation OP1 and the second operation OP2 is set as the first interval time TI1. For example, the pulse time TP may be not less than 100 ms and not more than 300 ms. On the other hand, the first interval time TI1 may be, for example, not less than 1 ms and not more than 50 ms. In the embodiments, the first interval time TI1 can be shortened. This is due to the phenomena that the heat capacity of the element portion 10E is small and a high speed temperature change can be obtained. Thereby, for example, fast detection can be performed.

For example, the first interval time TI1 between the first operation OP1 and the second operation OP2 may be ⅕ or less of the pulse time TP of the voltage VS applied to the conductive member 21 in the first operation OP1. The first interval time TI1 may be 1/10 or less of the pulse time TP. The first interval time TI1 may be 1/50 or less of the pulse time TP.

As shown in FIG. 6, a time when the third switch S3 shifts from the third connected state (ON) to the third disconnected state (OFF) in the first operation OP1 is set as a first time t1. In the second operation OP2, a time when the second switch S2 shifts from the second disconnected state (OFF) to the second connected state (ON) is set as a second time t2. A time duration from the first time t1 to the second time t2 corresponds to the first interval time TI1. A time duration when the third switch S3 is in the third connected state (ON) in the first operation OP1 corresponds to the pulse time TP. For example, the first interval time TI1 may be ⅕ or less of the pulse time TP.

As shown in FIG. 6, in this example, the third operation OP3 is performed after the second operation OP2. The time duration between the second operation OP2 and the third operation OP3 is defined as a second interval time TI2. The second interval time TI2 may be ⅕ or less of the pulse time TP. The second interval time TI2 may be 1/10 or less of the pulse time TP. The second interval time TI2 may be 1/50 or less of the pulse time TP. High-speed detection is possible.

In the sensor 110, the circuit section 70 may include the voltage source 76. The circuit section 70 may include the first current source 75a. The circuit section 70 may include the second current source 75b. At least one of the voltage source 76, the first current source 75a, and the second current source 75b may be provided separately from the sensor 110.

As shown in FIG. 2, at least a part of the circuit section 70 may be provided at the base 41. At least a part of the circuit section 70 may overlap the element portion 10E in a direction from the base 41 to the element portion 10E (for example, a Z-axis direction).

As shown in FIG. 1, the base 41 may include a semiconductor substrate 41s and an insulating layer 41i. The semiconductor substrate 41s may be, for example, a silicon substrate. The insulating layer 41i is provided on the semiconductor substrate 41s. A part of the semiconductor substrate 41s may include a transistor or the like. A part of the semiconductor substrate 41s may include a CMOS circuit. At least a part of the circuit section 70 may be formed by a part of the semiconductor substrate 41s.

### Second Embodiment

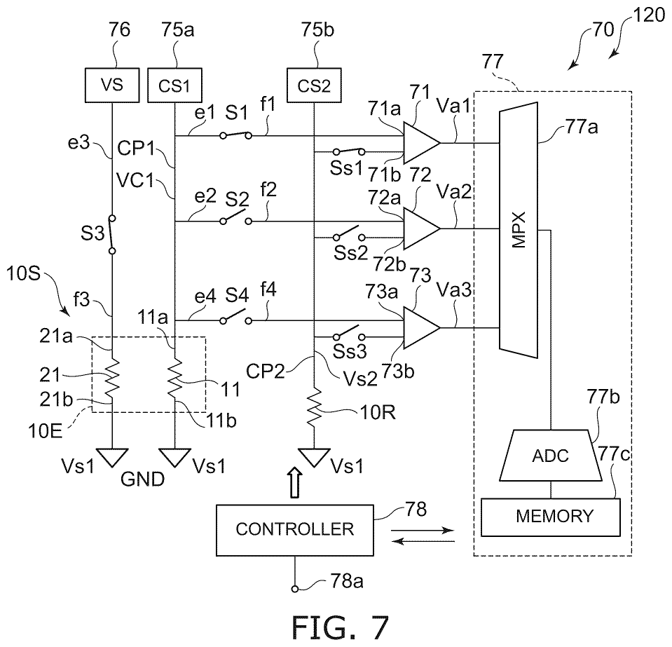

FIG. 7 is a block diagram illustrating a sensor according to a second embodiment.

As shown in FIG. 7, a sensor 120 according to the embodiment includes the sensor section 10S and the circuit section 70. The sensor section 10S includes the element portion 10E. The element portion 10E includes the sensor element 11 and the conductive member 21. The circuit section 70 includes the storage 77c and the controller 78. In the sensor 120, the configuration of the circuit section 70 is different from the configuration of the circuit section 70 in the sensor 110. The configuration of the sensor 120 other than this may be the same as the configuration of the sensor 110.

In the sensor 120, the sensor element end 11a of the sensor element 11 is electrically connected to the first current source 75a (CS1). The sensor element other end 11b of the sensor element 11 is set to the first reference voltage Vs1 (e.g., GND).

The controller 78 is configured to perform the first operation OP1 and the second operation OP2. The voltage at the first connection point CP1 between the sensor element end 11a and the first current source 75a is defined as the first connection point voltage VC1. In the first operation OP1, the controller 78 causes the storage 77c to store a first value Val corresponding to the first connection point voltage VC1 of the first connection point CP1 in a state where the voltage VS is applied to the conductive member 21.

In the second operation OP2, the controller 78 causes the storage 77c to store a second value Va2 corresponding to the first connection point voltage VC1 in a state where the voltage VS is not applied to the conductive member 21. The circuit section 70 is configured to output a value corresponding to the first difference between the first value Val and the second value Va2. For example, the value may be output from the output portion 78a of the controller 78 provided in the circuit section 70.

The value corresponding to the first difference between the first value Val and the second value Va2 corresponds to, for example, the concentration of the detection target gas. The value corresponding to the first difference may correspond to the type of detection target gas. In the embodiment, the detection result can be obtained with high accuracy using one sensor section 10S.

The controller 78 may be configured to perform the third operation OP3. In the third operation OP3, the controller 78 causes the storage 77c to store a third value Va3 corresponding to the first connection point voltage VC1 in a state where the voltage VS is not applied to the conductive member 21. The circuit section 70 is configured to output a correction value obtained by correcting the first difference based on the third value Va3. The correction value may be output from the output portion 78a of the controller 78, for example.

An example of the circuit section 70 in the sensor 120 will be described below.

As shown in FIG. 7, in this example, the circuit section 70 includes the first differential circuit 71, the second differential circuit 72, the first switch S1, the second switch S2, the third switch S3, and a first wiring switch Ss1, and a second wiring switch Ss2.

The first switch end e1 of the first switch S1 is electrically connected to the first connection point CP1. The first switch other end f1 of the first switch S1 is electrically connected to the first input end 71a of the first differential circuit 71. The first other input end 71b of the first differential circuit 71 is electrically connected to the second connection point CP2 of the second reference voltage Vs2 via the first wiring switch Ss1.

The second switch end e2 of the second switch S2 is electrically connected to the first connection point CP1. The second switch other end f2 of the second switch S2 is electrically connected to the second input end 72a of the second differential circuit 72. The second other input end 72b of the second differential circuit 72 is electrically connected to the second connection point CP2 via the second wiring switch Ss2.

The third switch end e3 of the third switch S3 is connected to the voltage source 76 (voltage VS). The third switch other end f3 of the third switch S3 is electrically connected to the conductive member end 21a of the conductive member 21. The conductive member other end 21b of the conductive member 21 is set to the first reference voltage Vs1 (e.g., GND).

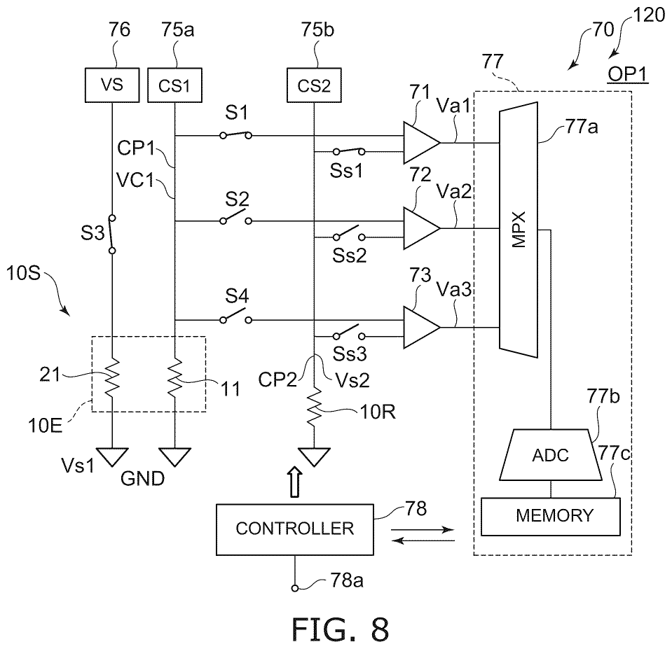

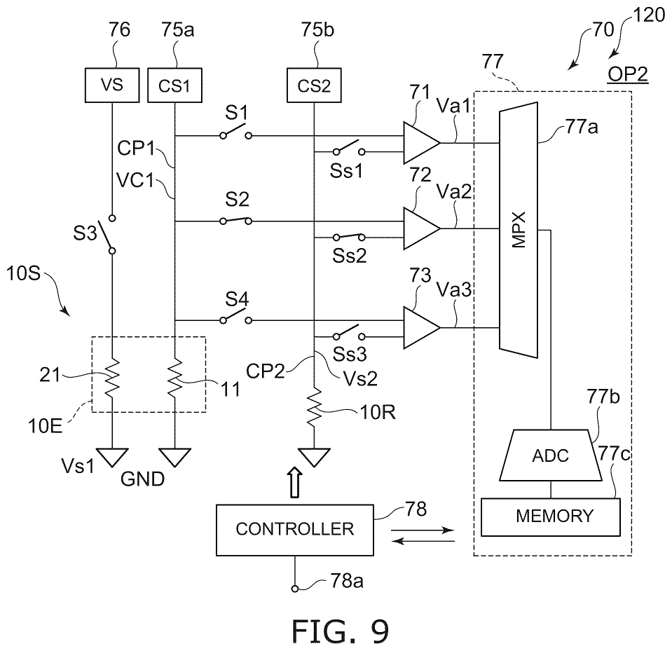

FIGS. 8 and 9 are block diagrams illustrating the operation of the sensor according to the second embodiment.

FIG. 8 illustrates the first operation OP1. FIG. 9 illustrates the second operation OP2.

As shown in FIG. 8, in the first operation OP1, the controller 78 sets the first switch S1 to the first connected state, sets the first wiring switch Ss1 to a first wiring connected state, sets the second switch S2 to the second disconnected state, sets the second wiring switch Ss2 to a second wiring disconnected state, and sets the third switch S3 to the third connected state. Thereby, the first connection point voltage VC1 when the voltage VS is applied to the conductive member 21 is inputted to the first differential circuit 71. The output of the first differential circuit 71 is stored in the storage 77c. In this example, the difference between the first connection point voltage VC1 and the second reference voltage Vs2 is output from the first differential circuit 71.

As shown in FIG. 9, in the second operation OP2, the controller 78 sets the first switch S1 to the first disconnected state, sets the first wiring switch Ss1 to the first wiring disconnected state, sets the second switch S2 to the second connected state, sets the second wiring switch Ss2 to the second wiring connected state, and sets the third switch S3 to the third disconnected state. Thereby, the first connection point voltage VC1 when the voltage VS is not applied to the conductive member 21 is inputted to the second differential circuit 72. The output of the second differential circuit 72 is stored in the storage 77c. In this example, the difference between the first connection point voltage VC1 and the second reference voltage Vs2 is output from the second differential circuit 72.

As shown in FIG. 8, the circuit section 70 may further include a third differential circuit 73, the fourth switch S4, and a third wiring switch Ss3. The fourth switch end e4 of the fourth switch S4 is electrically connected to the first connection point CP1. The fourth switch other end f4 of the fourth switch S4 is electrically connected to a third input end 73a of the third differential circuit 73. The third other input end 73b of the third differential circuit 73 is electrically connected to the second connection point CP2 via the third wiring switch Ss3. With such a configuration, the third operation OP3 may be performed.

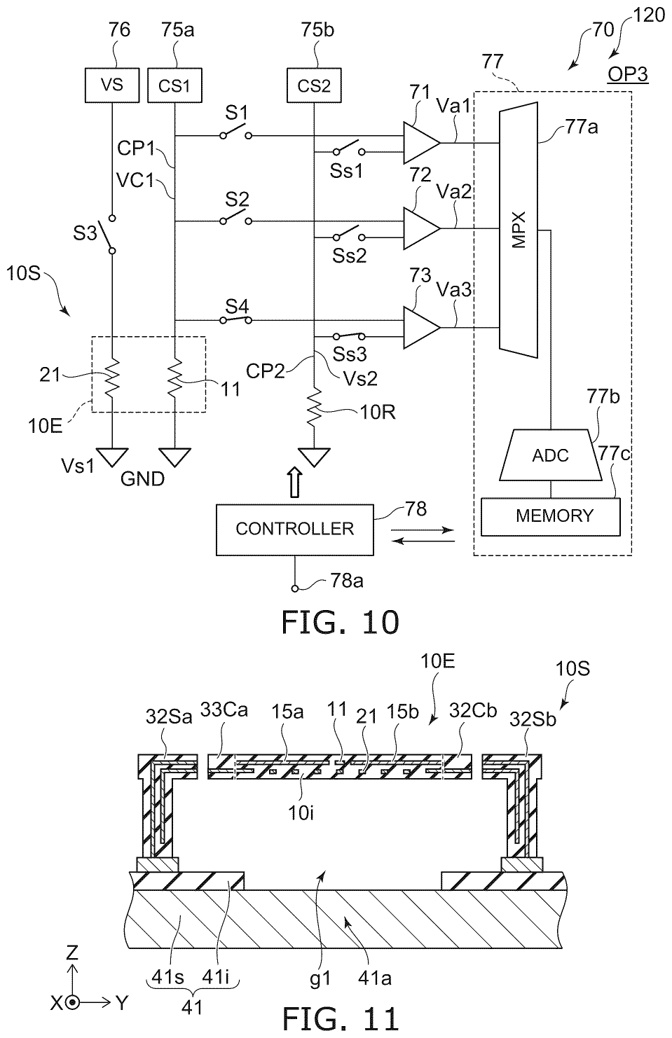

FIG. 10 is a block diagram illustrating the operation of the sensor according to the second embodiment.

FIG. 10 illustrates the third operation.

As previously described, in the third operation OP3, the controller 78 causes the storage 77c to store the third value Va3 corresponding to the first connection point voltage VC1 in the state in which the voltage VS is not applied to the conductive member 21. As shown in FIG. 10, for example, in the third operation OP3, the controller 78 sets the first switch S1 to the first disconnected state, the first wiring switch Ss1 to the first wiring disconnected state, the second switch S2 to the second disconnected state, the second wiring switch Ss2 to the second wiring disconnected state, the third switch S3 to the third connected state, the fourth switch S4 to the fourth connected state, and the third wiring switch Ss3 to the third wiring connected state.

Thereby, the first connection point voltage VC1 when the voltage VS is not applied to the conductive member 21 is inputted to the third differential circuit 73. The output of the third differential circuit 73 is stored in the storage 77c. In this example, the difference between the first connection point voltage VC1 and the second reference voltage Vs2 is output from the third differential circuit 73.

The sensor 120 may perform the operation described with reference to FIG. 6. For example, the first interval time TI1 between the first operation OP1 and the second operation OP2 may be ⅕ or less of the pulse time TP of the voltage VS applied to the conductive member 21 in the first operation OP1 (see FIG. 6). The first interval time TI1 may be 1/10 or less of the pulse time TP. The first interval time TI1 may be 1/20 or less of the pulse time TP.

As described with reference to FIG. 2, the sensor section 10S may include the base 41 and the first support portion 31Sa fixed to the base 41. The first support portion 31Sa supports the element portion 10E. The first gap g1 is provided between the base 41 and the element portion 10E. A small heat capacity is obtained and high-speed detection is possible.

At least a part of the circuit section 70 may be provided at the base 41. At least a part of the circuit section 70 may overlap the element portion 10E in the direction from the base 41 to the element portion 10E (see FIG. 2).

Hereinafter, an example of the sensor section 10S will be described.

FIG. 11 is a schematic cross-sectional view illustrating the sensor according to the embodiment.

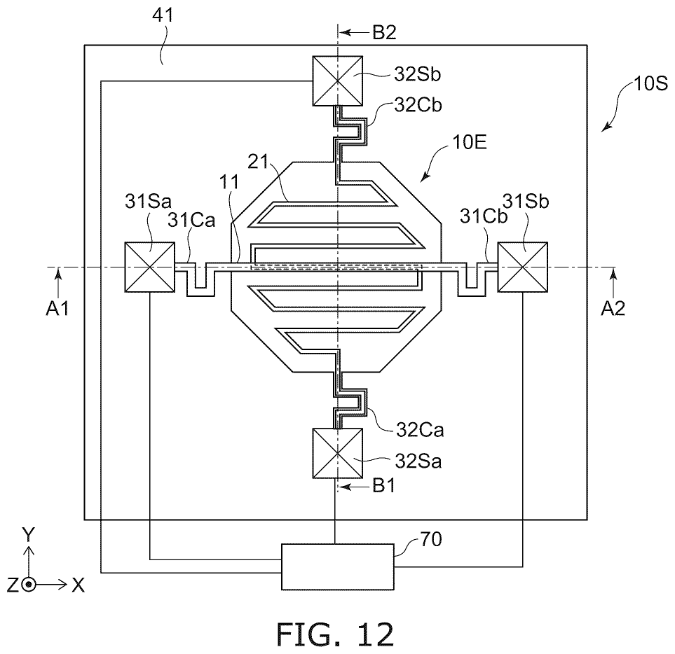

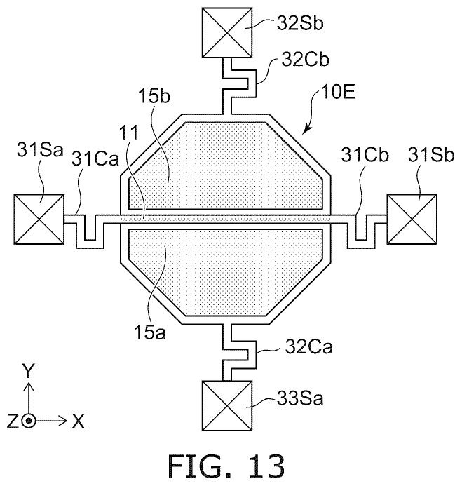

FIGS. 12 and 13 are schematic plan views illustrating the sensor according to the embodiment.

These figures illustrate the sensor section 10S. The configuration illustrated in these figures is applicable to the sensor 110 or the sensor 120. FIG. 2 corresponds to a cross-sectional view of the line A1-A2 of FIG. 12. FIG. 11 corresponds to a cross-sectional view of the line B1-B2 of FIG. 12. FIG. 12 corresponds to a plan view in a plane including the conductive member 21. FIG. 13 corresponds to a plan view in a plane including the sensor element 11.

As shown in FIG. 11, the sensor section 10S includes a second support portion 32Sa and a second opposing support portion 32Sb. The second support portion 32Sa is fixed to the base 41. The second support portion 32Sa supports the element portion 10E. The second opposing support portion 32Sb is fixed to the base 41. The second opposing support portion 32Sb support the element portion 10E.

As shown in FIG. 12, a first direction (e.g., X-axis direction) from the first support portion 31Sa to the first opposing support portion 31Sb crosses a second direction (e.g., Y-axis direction) from the second support portion 32Sa to the second opposing support portion 32Sb. A third direction (e.g., the Z-axis direction) from the base 41 to the element portion 10E crosses a plane including the first direction and the second direction.

As shown in FIGS. 11 and 12, in this example, the sensor section 10S includes a second connect portion 32Ca and a second opposing connect portion 32Cb. The second connect portion 32Ca is supported by the second support portion 32Sa and supports the element portion 10E. The second opposing connect portion 32Cb is supported by the second opposing support portion 32Sb and supports the element portion 10E. These connect potions may have a meandering structure.

As shown in FIG. 13, a first film 15a and a second film 15b may be provided. The sensor element 11 is provided between the first film 15a and the second film 15b. The materials of these films may be the same as the material of the sensor element 11. These films suppress deformation of the element portion 10E, for example.

The embodiments may include the following technical proposals.

### (Technical Proposal 1)

A sensor, comprising:
a sensor section including an element portion, the element portion including a sensor element and a conductive member; and
a circuit section including a first differential circuit, a voltage holding circuit, a first switch, a second switch, a third switch, and a controller;
a sensor element end of the sensor element being electrically connected to a first current source,
a sensor element other end of the sensor element being set to a first reference voltage,
a first switch end of the first switch being electrically connected to a first connection point between the sensor element end and the first current source,
a first switch other end of the first switch being electrically connected to an input end of the voltage holding circuit,
an output end of the voltage holding circuit being electrically connected to a first input end of the first differential circuit,
a second switch end of the second switch being electrically connected to the first connection point,
a second switch other end of the second switch being electrically connected to a first other input end of the first differential circuit,
a third switch end of the third switch being connected to a voltage source,
a third switch other end of the third switch being electrically connected to a conductive member end of the conductive member,
a conductive member other end of the conductive member being set to the first reference voltage,
the controller being configured to perform a first operation and a second operation,
in the first operation, the controller being configured to set the third switch to a third connected state, to set the first switch to a first connected state, and to set the second switch to a second disconnected state, the voltage holding circuit being configured to hold a first connection point voltage of the first connection point in the first operation, and
in the second operation, the controller being configured to set the third switch to a third disconnected state, to set the first switch to a first disconnected state, and to set the second switch to a second connected state, the first differential circuit being configured to output a first difference between the first connection point voltage in the second operation and a holding voltage held by the voltage holding circuit.

### (Technical Proposal 2)

The sensor according to Technical proposal 1, wherein
a first interval time between the first operation and the second operation is ⅕ or less of a pulse time of the voltage applied to the conductive member in the first operation.

### (Technical Proposal 3)

The sensor according to Technical proposal 1, wherein
the second operation is performed after the first operation,
a first interval time from a time when the third switch is shifted from the third connected state to the third disconnected state in the first operation to a time when the second switch is shifted from the second disconnected state to the second connected state in the second operation is ⅕ or less of a pulse time in which the third switch is in the third connection state in the first operation.

### (Technical Proposal 4)

The sensor according to Technical proposal 2 or 3, wherein
the first interval time is 1/10 or less of the pulse time.

### (Technical Proposal 5)

The sensor according to any one of Technical proposals 1-4, wherein
the first difference corresponds to a concentration of a detection target gas.

### (Technical Proposal 6)

The sensor according to any one of Technical proposals 1-5, wherein
the circuit section further includes at least one of the first current source or the voltage source.

### (Technical Proposal 7)

The sensor according to any one of Technical proposals 1-6, wherein
the circuit section further includes a second differential circuit and a fourth switch,
a fourth switch end of the fourth switch is electrically connected to the first connection point,
a fourth switch other end of the fourth switch is electrically connected to a second input end of the second differential circuit, and
a second other input end of the second differential circuit is set to a second reference voltage.

### (Technical Proposal 8)

The sensor according to Technical proposal 7, wherein
the controller is configured to further perform a third operation, and
in the third operation, the controller is configured to set the third switch to the third disconnected state, to set the first switch to the first disconnected state, to set the second switch to the second disconnected state, and to set the fourth switch to a fourth connected state, the second differential circuit is configured to output a second difference between the first connection point voltage in the third operation and the second reference voltage.

### (Technical Proposal 9)

The sensor according to Technical proposal 8, wherein
the circuit section is configured to output a value obtained by correcting the first difference based on the second difference.

### (Technical Proposal 10)

The sensor according to Technical proposal 8, wherein
the circuit section further includes a storage configured to store the first difference and the second difference,
the circuit section acquires the first difference and the second difference stored in the storage section, and
the circuit section is configured to output a value of the first difference corrected based on the second difference.

### (Technical Proposal 11)

The sensor according to any one of Technical proposals 7-10, wherein
the second other input end is electrically connected to a second connection point between a reference resistance element end of a reference resistance element and a second current source, and
a reference resistance element other end of the reference resistance element is set to the first reference voltage.

### (Technical Proposal 12)

The sensor according to any one of Technical proposals 1-11, wherein
the sensor section includes
   a base, and
   a first support portion fixed to the base,
the first support portion supports the element portion, and
a first gap is provided between the base and the element portion.

### (Technical Proposal 13)

The sensor according to Technical proposal 12, wherein
at least a part of the circuit section is provided at the base, and
the at least the part of the circuit portion overlaps the element portion in a direction from the base to the element portion.

### (Technical Proposal 14)

A sensor, comprising:
a sensor section including an element portion, the element portion including a sensor element and a conductive member; and
a circuit section including a storage and a controller,
a sensor element end of the sensor element being electrically connected to a first current source,
a sensor element other end of the sensor element being set to a first reference voltage,
the controller being configured to perform a first operation and a second operation,
in the first operation, the controller being configured to cause the storage to store a first value corresponding to a first connection point voltage of a first connection point between the sensor element end and the first current source in a state where a voltage is applied to the conductive member,
in the second operation, the controller being configured to cause the storage to stores a second value corresponding to the first connection point voltage in a state where the voltage is not applied to the conductive member, and
the circuit section being configured to output a value corresponding to a first difference between the first value and the second value.

### (Technical Proposal 15)

The sensor according to Technical proposal 14, wherein
the controller is configured to perform a third operation,
in the third operation, the controller is configured to cause the storage to store a third value corresponding to the first connection point voltage in a state where the voltage is not applied to the conductive member, and
the circuit section is configured to output a correction value obtained by correcting the first difference based on the third value.

### (Technical Proposal 16)

The sensor according to Technical proposal 14, wherein
the circuit section further includes a first differential circuit, a second differential circuit, a first switch, a second switch, a third switch, a first wiring switch and a second wiring switch,
a first switch end of the first switch is electrically connected to the first connection point,
a first switch other end of the first switch is electrically connected to a first input end of the first differential circuit,
a first other input end of the first differential circuit is electrically connected to a second connection point of a second reference voltage via the first wiring switch,
a second switch end of the second switch is electrically connected to the first connection point,
the second switch other end of the second switch is electrically connected to a second input end of the second differential circuit,
the second other input end of the second differential circuit is electrically connected to the second connection point via the second wiring switch,
a third switch end of the third switch is connected to a voltage source,
a third switch other end of the third switch is electrically connected to a conductive member end of the conductive member,
a conductive member other end of the conductive member is set to the first reference voltage,
in the first operation, the controller is configured to set the first switch to a first connected state, to set the first wiring switch to a first wiring connected state, to set the second switch to a second disconnected state, to set the second wiring switch to a second wiring disconnected state, and to set the third switch to a third connected state, and
in the second operation, the controller is configured to set the first switch to a first disconnected state, to set the first wiring switch to a first wiring disconnected state, to set the second switch to a second connected state, to set the second wiring switch to the second wiring connected state, and to set the third switch to a third disconnected state.

### (Technical Proposal 17)

The sensor according to Technical proposal 16, wherein
the controller is configured to perform a third operation,
in the third operation, the controller is configured to cause the storage to stores a third value corresponding to the first connection point voltage in a state where the voltage is not applied to the conductive member,
the circuit section further includes a third differential circuit, a fourth switch and a third wiring switch,
a fourth switch end of the fourth switch is electrically connected to the first connection point,
a fourth switch other end of the fourth switch is electrically connected to a third input end of the third differential circuit,
a third other input end of the third differential circuit is electrically connected to the second connection point via the third wiring switch, and
in the third operation, the controller is configured to set the first switch to the first disconnected state, to set the first wiring switch to the first disconnected state, to set the second switch to the second disconnected state, to set the third switch to the third connected state, to set the fourth switch to a fourth connected state, and to set the third wiring switch to a third wiring connected state.

### (Technical Proposal 18)

The sensor according to any one of Technical proposals 14-17, wherein
a first interval time between the first operation and the second operation is ⅕ or less of a pulse time of the voltage applied to the conductive member in the first operation.

### (Technical Proposal 19)

The sensor according to any one of Technical proposals 14-18, wherein
the sensor section includes
   a base body, and
   a first support portion fixed to the base,
the first support portion supports the element portion, and
a first gap is provided between the base and the element portion.

### (Technical Proposal 20)

The sensor according to Technical proposal 19, wherein
at least a part of the circuit section is provided at the base, and
the at least the part of the circuit section overlaps the element portion in a direction from the base to the element portion.

According to the embodiment, a sensor with improved characteristics can be provided.

Hereinabove, exemplary embodiments of the invention are described with reference to specific examples. However, the embodiments of the invention are not limited to these specific examples. For example, one skilled in the art may similarly practice the invention by appropriately selecting specific configurations of components included in the sensors such as bases, sensor sections, controllers, etc., from known art. Such practice is included in the scope of the invention to the extent that similar effects thereto are obtained.

Further, any two or more components of the specific examples may be combined within the extent of technical feasibility and are included in the scope of the invention to the extent that the purport of the invention is included.

Moreover, all sensors practicable by an appropriate design modification by one skilled in the art based on the sensors described above as embodiments of the invention also are within the scope of the invention to the extent that the purport of the invention is included.

Various other variations and modifications can be conceived by those skilled in the art within the spirit of the invention, and it is understood that such variations and modifications are also encompassed within the scope of the invention.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the invention.

What is claimed is:

1. A sensor, comprising:
a sensor section including an element portion, the element portion including a sensor element and a conductive member; and
a circuit section including a first differential circuit, a voltage holding circuit, a first switch, a second switch, a third switch, and a controller;
a sensor element end of the sensor element being electrically connected to a first current source,
a sensor element other end of the sensor element being set to a first reference voltage,
a first switch end of the first switch being electrically connected to a first connection point between the sensor element end and the first current source,
a first switch other end of the first switch being electrically connected to an input end of the voltage holding circuit,
an output end of the voltage holding circuit being electrically connected to a first input end of the first differential circuit,
a second switch end of the second switch being electrically connected to the first connection point,
a second switch other end of the second switch being electrically connected to a first other input end of the first differential circuit,
a third switch end of the third switch being connected to a voltage source,
a third switch other end of the third switch being electrically connected to a conductive member end of the conductive member,
a conductive member other end of the conductive member being set to the first reference voltage,
the controller being configured to perform a first operation and a second operation,
in the first operation, the controller being configured to set the third switch to a third connected state, to set the first switch to a first connected state, and to set the second switch to a second disconnected state, the voltage holding circuit being configured to hold a first connection point voltage of the first connection point in the first operation, and
in the second operation, the controller being configured to set the third switch to a third disconnected state, to set the first switch to a first disconnected state, and to set the second switch to a second connected state, the first differential circuit being configured to output a first difference between the first connection point voltage in the second operation and a holding voltage held by the voltage holding circuit.

2. The sensor according to claim 1, wherein
a first interval time between the first operation and the second operation is ⅕ or less of a pulse time of the voltage applied to the conductive member in the first operation.

3. The sensor according to claim 1, wherein
the second operation is performed after the first operation,
a first interval time from a time when the third switch is shifted from the third connected state to the third disconnected state in the first operation to a time when the second switch is shifted from the second disconnected state to the second connected state in the second operation is ⅕ or less of a pulse time in which the third switch is in the third connection state in the first operation.

4. The sensor according to claim 2, wherein
the first interval time is 1/10 or less of the pulse time.

5. The sensor according to claim 1, wherein
the first difference corresponds to a concentration of a detection target gas.

6. The sensor according to claim 1, wherein
the circuit section further includes at least one of the first current source or the voltage source.

7. The sensor according to claim 1, wherein
the circuit section further includes a second differential circuit and a fourth switch,
a fourth switch end of the fourth switch is electrically connected to the first connection point,
a fourth switch other end of the fourth switch is electrically connected to a second input end of the second differential circuit, and
a second other input end of the second differential circuit is set to a second reference voltage.

8. The sensor according to claim 7, wherein
the controller is configured to further perform a third operation, and
in the third operation, the controller is configured to set the third switch to the third disconnected state, to set the first switch to the first disconnected state, to set the second switch to the second disconnected state, and to set the fourth switch to a fourth connected state, the second differential circuit is configured to output a second difference between the first connection point voltage in the third operation and the second reference voltage.

9. The sensor according to claim 8, wherein
the circuit section is configured to output a value obtained by correcting the first difference based on the second difference.

10. The sensor according to claim 8, wherein
the circuit section further includes a storage configured to store the first difference and the second difference,
the circuit section acquires the first difference and the second difference stored in the storage section, and
the circuit section is configured to output a value of the first difference corrected based on the second difference.

11. The sensor according to claim 7, wherein
the second other input end is electrically connected to a second connection point between a reference resistance element end of a reference resistance element and a second current source, and
a reference resistance element other end of the reference resistance element is set to the first reference voltage.

12. The sensor according to claim 1, wherein
the sensor section includes
a base, and
a first support portion fixed to the base,
the first support portion supports the element portion, and
a first gap is provided between the base and the element portion.

13. The sensor according to claim 12, wherein
at least a part of the circuit section is provided at the base, and
the at least the part of the circuit portion overlaps the element portion in a direction from the base to the element portion.

14. A sensor, comprising:
a sensor section including an element portion, the element portion including a sensor element and a conductive member; and
a circuit section including a storage and a controller,
a sensor element end of the sensor element being electrically connected to a first current source,
a sensor element other end of the sensor element being set to a first reference voltage,
the controller being configured to perform a first operation and a second operation,
in the first operation, the controller being configured to cause the storage to store a first value corresponding to a first connection point voltage of a first connection point between the sensor element end and the first current source in a state where a voltage is applied to the conductive member,
in the second operation, the controller being configured to cause the storage to stores a second value corresponding to the first connection point voltage in a state where the voltage is not applied to the conductive member, and
the circuit section being configured to output a value corresponding to a first difference between the first value and the second value.

15. The sensor according to claim 14, wherein
the controller is configured to perform a third operation,
in the third operation, the controller is configured to cause the storage to store a third value corresponding to the first connection point voltage in a state where the voltage is not applied to the conductive member, and
the circuit section is configured to output a correction value obtained by correcting the first difference based on the third value.

16. The sensor according to claim 14, wherein
the circuit section further includes a first differential circuit, a second differential circuit, a first switch, a second switch, a third switch, a first wiring switch and a second wiring switch,
a first switch end of the first switch is electrically connected to the first connection point,
a first switch other end of the first switch is electrically connected to a first input end of the first differential circuit,
a first other input end of the first differential circuit is electrically connected to a second connection point of a second reference voltage via the first wiring switch,
a second switch end of the second switch is electrically connected to the first connection point,
the second switch other end of the second switch is electrically connected to a second input end of the second differential circuit,
the second other input end of the second differential circuit is electrically connected to the second connection point via the second wiring switch,
a third switch end of the third switch is connected to a voltage source,
a third switch other end of the third switch is electrically connected to a conductive member end of the conductive member,
a conductive member other end of the conductive member is set to the first reference voltage,
in the first operation, the controller is configured to set the first switch to a first connected state, to set the first wiring switch to a first wiring connected state, to set the second switch to a second disconnected state, to set the second wiring switch to a second wiring disconnected state, and to set the third switch to a third connected state, and
in the second operation, the controller is configured to set the first switch to a first disconnected state, to set the first wiring switch to a first wiring disconnected state, to set the second switch to a second connected state, to set the second wiring switch to the second wiring connected state, and to set the third switch to a third disconnected state.

17. The sensor according to claim 16, wherein
the controller is configured to perform a third operation,
in the third operation, the controller is configured to cause the storage to stores a third value corresponding to the first connection point voltage in a state where the voltage is not applied to the conductive member,
the circuit section further includes a third differential circuit, a fourth switch and a third wiring switch,
a fourth switch end of the fourth switch is electrically connected to the first connection point,
a fourth switch other end of the fourth switch is electrically connected to a third input end of the third differential circuit,
a third other input end of the third differential circuit is electrically connected to the second connection point via the third wiring switch, and
in the third operation, the controller is configured to set the first switch to the first disconnected state, to set the first wiring switch to the first disconnected state, to set the second switch to the second disconnected state, to set the third switch to the third connected state, to set the fourth switch to a fourth connected state, and to set the third wiring switch to a third wiring connected state.

18. The sensor according to claim 14, wherein
a first interval time between the first operation and the second operation is ⅕ or less of a pulse time of the voltage applied to the conductive member in the first operation.

19. The sensor according to claim 14, wherein
the sensor section includes
a base body, and
a first support portion fixed to the base,
the first support portion supports the element portion, and
a first gap is provided between the base and the element portion.

20. The sensor according to claim 19, wherein
at least a part of the circuit section is provided at the base, and
the at least the part of the circuit section overlaps the element portion in a direction from the base to the element portion.
